(19) United States
(12) Patent Application Publication
(10) Pub. No.: US 2010/0324509 A1
(43) Pub. Date: Dec. 23, 2010

(54) Face Mask

(76) Inventor: Tina LEE, Taipei (TW)

Correspondence Address: NIKOLAI & MERSEREAU, P.A., 900 SECOND AVENUE SOUTH, SUITE 820, MINNEAPOLIS, MN, 55402, US

(21) Appl. No.: 12/489,927

(22) Filed: Jun. 23, 2009

(51) Int. Cl. A61M 37/00

(52) U.S. Cl. 604/303

(57) ABSTRACT

A face mask has a substrate and a carrier. The substrate has multiple resilient units and a face portion. The resilient units makes the substrate extendable. The face portion corresponds to the face and has three holes and two ear slits. The holes are defined through the face portion correspond to and are mounted around eyes and mouth of a user. Two ear slits are defined through the face portion corresponding to and hung on ears of the user. The carrier is impregnated in the substrate and may be a medicinal herb extract.

### BACKGROUND OF THE INVENTION

1. Field of the Invention

The present invention relates a face mask, and more particularly to a face mask that can cover the face including a jaw of a user and be hung from ears of the user.

2. Description of Related Art

With reference to FIG. 3, a face mask is a popular cosmetic product and is usually impregnated with a carrier having cream, moisturizer or the like dissolved therein. The face mask is placed on a face to allow the creams are absorbed by the skin. However, disadvantages of the conventional face mask follow.

1. The conventional face mask is weakly elastic, so cannot be stretched to fit different facial configurations of different users so is produced oversized and folded causing air pockets to be formed between face and mask.

2. The conventional face mask does not cover a jaw of the user.

3. The conventional face mask cannot be hung from the ears of the user, so the face mask easily falls off from the face.

To overcome these shortcomings, the present invention provides a face mask to mitigate or obviate the aforementioned problems.

### SUMMARY OF THE INVENTION

A face mask has a substrate and a carrier. The substrate has multiple resilient units and a face portion. The resilient units makes the substrate extendable. The face portion corresponds to the face and has three holes and two ear slits. The holes are defined through the face portion correspond to and are mounted around eyes and mouth of a user. Two ear slits are defined through the face portion corresponding to and hung on ears of the user. The carrier is impregnated in the substrate and may be a medicinal herb extract.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a face mask in accordance with the present invention;

FIG. 2 is a series of operational perspective views of the face mask in FIG. 1 being worn; and

FIG. 3 is a perspective view of a conventional face mask in accordance with the prior art.

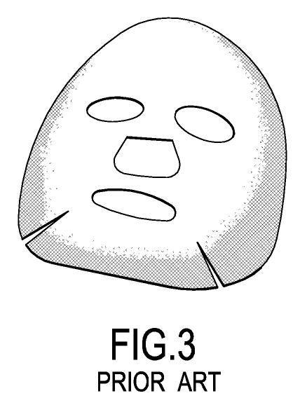

### DETAILED DESCRIPTION OF PREFERRED EMBODIMENT

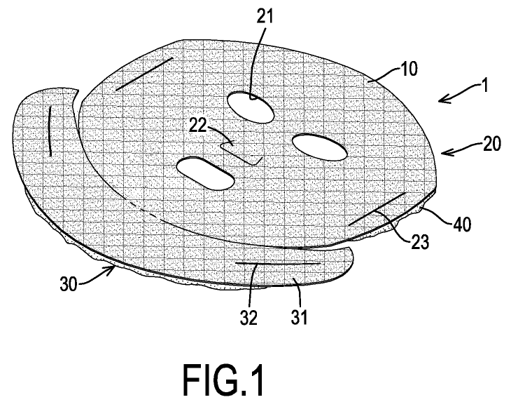

With reference to FIG. 1, a face mask (1) in accordance with the present invention is non-woven material and comprises a substrate (20) and a carrier (40).

The substrate (20) has multiple resilient units (10), a face portion and a jaw portion (30). The resilient units (10) may be grooves, dot holes or the like to allow the substrate (20) to be extended. The face portion may be an ellipse, corresponds to a face and has three holes (21), a nose sheet (22), a central segment, two ear slits (23), two sides and a bottom edge. The holes (21) are defined through the face portion corresponding respectively to human's eyes and mouth of the face. The nose sheet (22) is formed on the central segment of the face portion and corresponds in shape to the human's nose. The ear slits (23) are defined through the face portion respectively adjacent to the two sides of the face portion and correspond respectively to the human's ears. The jaw portion (30) may be an arc and corresponds to an underside of a jaw, is connected to the bottom edge of the face portion and comprises two hanging parts (31) and two ends. The hanging parts (31) are formed on and extend from the ends of the jaw portion (30) and each comprises a hanging slit (32). The hanging slit (32) is defined through the hanging part (31).

The carrier (40) may be a medicinal herb extract, is impregnated in the substrate (20) to supply nutrients to skin.

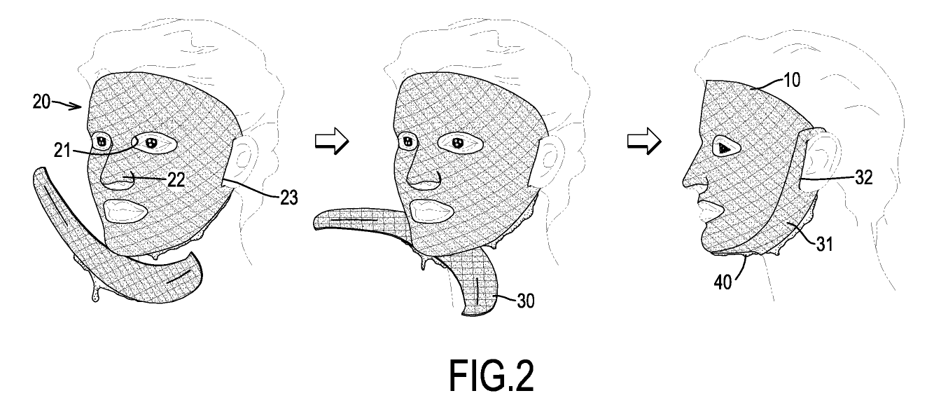

With further reference to FIG. 2, the face mask (1) is used to cover the face of the user. The holes (21) correspond to and are mounted around the eyes and mouth of the user. The nose sheet (22) corresponds to and covers the nose of the user and allows the user to breath during use of the face mask (1). The ears slits (23) are hung from the ears, and the two hanging parts (31) are pulled to be hung on the ears using the hanging slits (32) in the hanging parts (31).

With the arrangement of the resilient units (10) on the substrate (20), the face mask (1) is extendable to fit with different kinds of facial configurations. Furthermore, the jaw portion (30) can cover and provide the carrier (40) to the skin and the face mask (1) is versatile in use. Additionally, with the hanging parts (31) hung on the ears, the face mask (1) can be kept from falling off from the face of the user.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A face mask comprising:
a substrate having
multiple resilient units formed on the substrate to make the substrate extendable;
a face portion having
three holes defined through the face portion for corresponding to and mounted around eyes and mouth of a user;
two ear slits defined through the face portion for corresponding to and mounted around ears of the user;
a bottom edge;
a central segment; and
a carrier impregnated in the substrate.

2. The face mask as claimed in claim 1, wherein the face mask further has a jaw portion connected to the bottom edge of the face portion.

3. The face mask as claimed in claim 2, wherein the jaw portion further has
two ends; and
two hanging parts formed on and extended from the ends of the jaw portion and each having a hanging slit.

4. The face mask as claimed in claim 1, wherein the resilient units of the substrate are squares.

5. The face mask as claimed in claim 2, wherein the resilient units of the substrate are squares.

6. The face mask as claimed in claim 3, wherein the resilient units of the substrate are squares.

7. The face mask as claimed in claim 1, wherein the face portion further has a nose sheet formed on the central segment of the face portion.

8. The face mask as claimed in claim 6, wherein the face portion further has a nose sheet formed on the central segment of the face portion.

9. The face mask as claimed in claim 1, wherein the carrier is a medicinal herb extract.

10. The face mask as claimed in claim 2, wherein the carrier is a medicinal herb extract.
